(19) United States
(12) Patent Application Publication
(10) Pub. No.: US 2003/0092695 A1
(43) Pub. Date: May 15, 2003

(54) Metal salts of 3-methyl-chromane or thiochromane derivatives

(76) Inventors: Jaechon Jo, Seoul (KR); SungDae Park, Seoul (KR); HyunSuk Lim, Kyunggi-do (KR); SungOh Ahn, Seoul (KR); Kazumi Morikawa, Shizuoka (JP); Yoshitake Kanbe, Shizuoka (JP); Masahiro Nishimoto, Shizuoka (JP); MyungHwa Kim, Shizuoka (JP)

Correspondence Address: BROWDY AND NEIMARK, P.L.L.C., 624 NINTH STREET, NW, SUITE 300, WASHINGTON, DC, 20001-5303, US

(21) Appl. No.: 10/149,754

(22) PCT Filed: Dec. 13, 2000
(86) PCT No.: PCT/KR00/01445

(30) Foreign Application Priority Data
Dec. 13, 1999 (KR) 57066/1999

(51) Int. Cl. A61K 31/555; C07D 335/06; C07D 311/74; A61K 31/353; A61K 31/382

(52) U.S. Cl. 514/185; 514/432; 514/456; 549/23; 549/398

(57) ABSTRACT

The present invention relates to metal salts of 3-methyl-chromane or thiochromane derivatives, stereoisomers or hydrates thereof, and an anti-estrogenic pharmaceutical composition which comprises the above compound as an active component and exhibits a highly improved solubility.

### TECHNICAL FIELD

The present invention relates to metal salts of 3-methyl-chromane or thiochromane derivatives having anti-estrogenic activity More specifically, the present invention relates to metal salts of 3-methyl-chromane or thiochromane derivatives represented by the following formula (1):

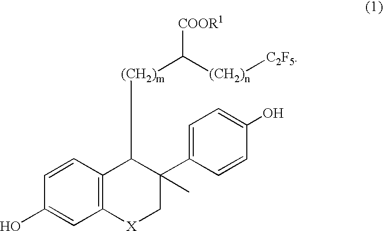

in which

X represents O or S,

R¹ represents metal,

m represents an integer of 2 to 14, and

n represents an integer of 2 to 7, stereoisomers or hydrates thereof, and an anti-estrogenic pharmaceutical composition which comprises the compound of formula (1) as an active component and exhibits a highly improved solubility.

### BACKGROUND ART

In treating diseases which are dependent upon a certain sexual hormone such as estrogen, it is important to significantly reduce or inhibit the effect induced by the hormone. For this purpose, it is desirable to reduce the level of hormone capable of acting on the receptor site which can be stimulated by sexual steroidal hormone. For instance, hysterectomy may be applied to limit the production of estrogen to the amount less than required to activate the receptor site. However, this method could not sufficiently inhibit the effect induced through the estrogen receptor. Practically, even when estrogen is completely absent, some of the receptors may be activated. Accordingly, it was considered that antagonists for estrogen can provide better therapeutic effect in comparison to the method for blocking only the production of sexual steroidal hormone (see, WO 96/26201). Thus, numerous anti-estrogenic compounds have been developed. For example, many patent publications including U.S. Pat. Nos. 4,760,061, 4,732,912, 4,904,661, 5,395,842 and WO 96/22092, etc. disclose various anti-estrogenic compounds. Sometimes, however, prior antagonists may act themselves as agonists, and therefore, activate rather than block the receptor. For example, Tamoxifen has been most widely used as an anti-estrogenic agent. However, it has a disadvantage that it exhibits estrogenic activity in some organs (see, M. Harper and A. Walpole, J. Reprod. Fertil., 1967, 13, 101).

As another non-steroidal anti-estrogenic compound, WO 93/10741 discloses a benzopyran derivative having aminoethoxyphenyl substituent (Endorecherche), the typical compound of which is EM-343 having the following structure:

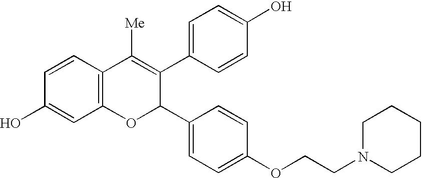

Therefore, it is required to develop an anti-estrogenic compound which has substantially or completely no agonistic effect and can effectively block the estrogenic receptor.

In addition, it has been known that 7α-substituted derivatives of estradiol, for example, 7α-(CH₂)₁₀CONBuMe derivatives, are steroidal anti-estrogenic agent without agonistic effect (see, EP Appl. 0138504, U.S. Pat. No. 4,659,516). Further, estradiol derivative having 7α-(CH₂)₉SOC₅H₆F₅ substituent has also been disclosed (see, Wakeling et al., Cancer Res., 1991, 51, 3867).

Non-steroidal anti-estrogenic drug without agonistic effect has been first reported by Wakeling et al. in 1987 (see, A. Wakeling and J. Bowler, J. Endocrinol., 1987, 112, R7). Meanwhile, U.S. Pat. No. 4,904,661 (ICI, Great Britain) discloses a phenol derivative having anti-estrogenic activity. This phenol derivative mainly has a tetrahydronaphthalene structure and includes, typically, the following compounds:

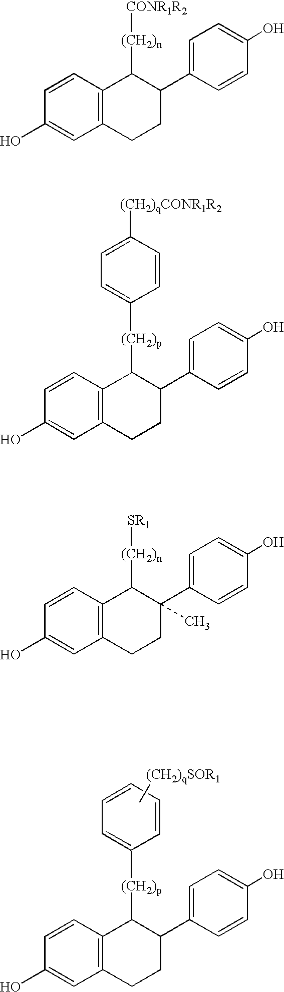

in which R₁, R₂, n, p and q are defined as described in the prior arts as mentioned above.

Some chromane and thiochromane derivatives have been reported as anti-estrogenic compounds having no agonistic effect (WO 98/25916). Although the existing anti-estrogenic compounds having no agonistic effect show a substantial therapeutic effect when administered via intravenous or subcutaneous injection, they show little therapeutic activity when administered orally, which is considered to be caused by several factors, one of which is the low bioavailability. Therefore, for convenience' sake in the case of administration, it is desired to develop anti-estrogenic compounds which show a sufficient effect when administered orally and at the same time have no agonistic effect.

Under these technical background, the present inventors have screened the anti-estrogenic activity of compounds having various structures. As a result, we have identified that 3-methyl-chromane or thiochromane derivatives represented by the following formula (1) can exhibit a good anti-estrogenic activity with no substantial agonistic effect even when orally administered. We also identified that if said compounds are converted to metal salts thereof, their physico-chemical properties including solubility can be highly improved and thus, can be used as a very excellent drug, whereby we completed the present invention.

### DISCLOSURE OF THE INVENTION

Therefore, the present invention relates to metal salts of 3-methyl-chromane or thiochromane derivatives represented by the following formula (1):

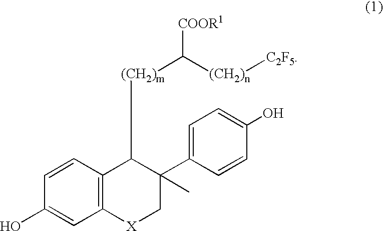

in which

X represents O or S,

R¹ represents metal,

m represents an integer of 2 to 14, and

n represents an integer of 2 to 7, stereoisomers or hydrates thereof.

It is also an object of the present invention to provide a medicine, more specifically an anti-estrogenic pharmaceutical composition which comprises the compound of formula (1) as an active component together with pharmaceutically acceptable carriers.

### BEST MODE FOR CARRYING OUT THE INVENTION

In the compound of formula (1) according to the present invention, the metal in the definition of R¹ includes alkali metals such as natrium, kalium, etc.; alkaline earth metals such as magenesium, calcium, etc.; rare earth metals such as cerium, samarium, etc.; and zinc, tin, etc. When R¹ is a monovalent metal such as an alkali metal, the metal combines with the residue of the compound of formula (1) in a ratio of 1:1. However, when R¹ is not a monovalent metal, it combines in a ratio of more than 1:1 depending on the valency of the metal.

The compound of formula (1) according to the present invention can exist as a stereoisomer, and thus, the present invention also includes each of the stereoisomers and their mixtures including racemate. Among the stereoisomers, compounds wherein the configuration of 3- and 4-position chiral carbons in the chromane (or thiochromane) ring is (3R, 4R) or (3S, 4S) or mixtures thereof are preferable, and in this case, compounds wherein the chiral carbon of the 4-position side chain of chromane (or thiochromane) ring, to which R¹OOC— group is attached, has the configuration of R or S or mixtures thereof are preferable.

Among the compound of formula (1), the preferred compounds include those wherein R¹ is alkali metal or alkaline earth metal, X is oxygen or sulfur, m is an integer of 6 to 10, and n is an integer of 3 to 5. Particularly preferred compounds include those wherein R¹ is alkali metal (particularly, natrium or kalium), alkaline earth metal (particularly calcium), m is an integer of 8 or 9.

As typical examples of the compound of formula (1), the following compounds can be mentioned:

Sodium (3′RS,4′RS)-10-[7-hydroxy-3-(4-hydroxyphenyl)-3-methylthiochroman-4-yl]-2-(4,4,5,5,5-pentafluoropentyl)decanoate;

Sodium (3′RS,4′RS)-11-[7-hydroxy-3-(4-hydroxyphenyl)-3-methylthiochroman-4-yl]-2-(4,4,5,5,5-pentafluoropentyl)undecanoate; and

Sodium (3′RS,4′RS)-11-[7-hydroxy-3-(4-hydroxyphenyl)-3-methylchroman-4-yl]-2-(4,4,5,5,5-pentafluoropentyl)undecanoate.

Metal salts of the compound of formula (1) show a superior solubility to the free compound. As can be seen from the following results of Experiments, the sodium salt compound of Example 1 of the present invention exhibits the same level of anti-estrogenic activity (see, Table 1) and simultaneously a highly improved solubility as compared with the corresponding free compound (see, Table 2). Particularly in the aspect of solubility, when the test is carried out using an artificial intestinal juice (FaSSIF) as the solvent, the metal salt compound is observed to have a several to scores of improved solubility over the free compound. Further, when water is used as the solvent, the metal salt compound shows the same degree of excellent solubility as the artificial intestinal juice case, whereas the free compound can hardly be solved in water. Therefore, it can be identified from the above results that the expected object of the present invention to effectively use the compound as an agent by changing the important physico-chemical properties such as solubility is satisfactorily achieved.

The compound of formula (1) according to the present invention can be prepared by the following Methods I to V, and thus, the present invention also provides these processes.

(Process I)

The compound of formula (1) can be prepared by a process characterized in that

(a) a compound of the following formula (2):

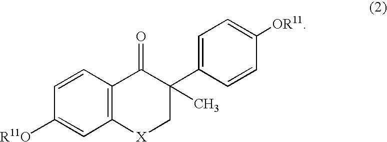

in which

X is defined as previously described, and

R¹¹ represents hydroxy or carboxy-protecting group, preferably t-butyldimethylsilyl, triisopropylsilyl, triethylsilyl, t-butyldiphenylsilyl, methoxymethyl, tetrahydropyranyl, methyl, ethyl, etc., is reacted with an acetylene compound of the following formula (3):

HC≡C—(CH₂)ₘ_{₁}OR¹² (3).

in which

m₁ represents a number of m−2, and

R¹² represents hydroxy or carboxy-protecting group, preferably t-butyldimethylsilyl, triisopropylsilyl, triethylsilyl, t-butyldiphenylsilyl, methoxymethyl, tetrahydropyranyl, methyl, ethyl, etc., in an inert solvent in the presence of a base to give a compound of the following formula (4):

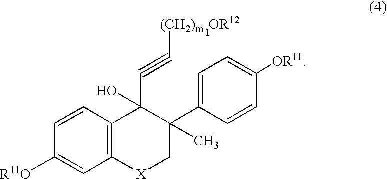

in which X, m₁, R¹¹ and R¹² are defined as previously described (where tetrahydrofuran, dioxane, dichloromethane or chloroform, preferably tetrahydrofuran or dioxane is used as the inert solvent, and n-butyllithium, sec-butyllithium or sodium hydride is used as the base, and the reaction is carried out at temperatures ranging from −78° C. to the boiling point of the reaction mixture, preferably from −78° C. to room temperature);

(b) the compound of formula (4) is reduced by sodium cyanoborohydride in an inert solvent in the presence of a Lewis acid to give a compound of the following formula (5):

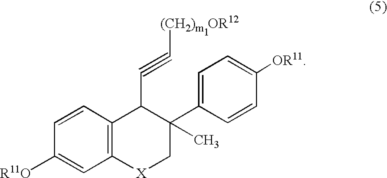

in which X, m₁, R¹¹ and R¹² are defined as previously described (where tetrahydrofuran, dioxane, dichloromethane, dichloroethane, or chloroform, preferably dichloroethane is used as the inert solvent, and zinc iodide is used as the Lewis acid, and the reaction is carried out at temperatures ranging from −78° C. to the boiling point of the reaction mixture, preferably from 0° C. to room temperature);

(c) the compound of formula (5) is catalytically hydrogenated in an inert solvent and optionally in the presence of sodium hydrogen carbonate to give a compound of the following formula (6):

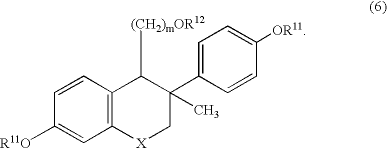

in which X, m, R¹¹ and R¹² are defined as previously described (where methanol, ethanol, ethyl acetate, tetrahydrofuran, dioxane, dichloromethane, dichloroethane, or chloroform, preferably tetrahydrofuran or ethyl acetate is used as the inert solvent, and activated Pd/C, palladium hydroxide or platinum oxide is used as the catalyst, and the reaction is carried out at temperatures ranging from room temperature to the boiling point of the reaction mixture, preferably at room temperature), however, the compound of formula (6) may be directly obtained from the compound of formula (4) through a catalytic hydrogenation reaction in an inert solvent (where the reaction conditions are the same as the step of preparing the compound of formula (6) from the compound of formula (5));

(d) the hydroxy group in the compound of formula (6) is deprotected by the treatment with one or more substances selected from a group consisting of tetrabutylammonium fluoride, cesium fluoride, hydrofluoride-pyridine, hydrochloride, sulfuric acid and p-toluenesulfonic acid in an inert solvent to give a compound of the following formula (7):

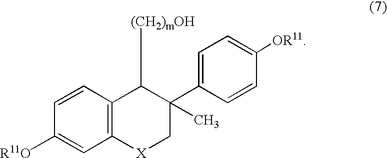

in which X, m, and R¹¹ are defined as previously described (where tetrahydrofuran, dioxane, dichloromethane, dichloroethane, or chloroform, preferably tetrahydrofuran is used as the inert solvent, and the reaction is carried out at temperatures ranging from room temperature to the boiling point of the reaction mixture);

(e) the compound of formula (7) is treated with methyl sulfonyl chloride or p-toluene sulfonyl chloride in an inert solvent in the presence of an organic base to change the group of (CH₂)ₘOH in compound (7) to a group of (CH₂)ₘO—SO₂CH₃ or (CH₂)ₘO—SO₂—C₆H₄-p-CH₃ (where tetrahydrofuran, dioxane, dichloromethane, dichloroethane, or chloroform, preferably dichloromethane is used as the inert solvent, and triethylamine or pyridine is used as the organic base, and the reaction is carried out at temperatures ranging from room temperature to the boiling point of the reaction mixture, preferably at room temperature), or the resulting compound is further treated with metal halide in an inert solvent to give a compound of the following formula (8):

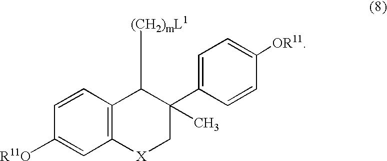

in which

X, m, and R¹¹ are defined as previously described, and

L¹ represents a leaving group, preferably methylsulfonyloxy, p-toluenesulfonyloxy, halogen, etc. (where acetone, tetrahydrofuran, dioxane, dichloromethane, dichloroethane or chloroform, preferably dichloromethane is used as the inert solvent, and sodium iodide or potassium iodide is used as the metal halide, the reaction is carried out at temperatures ranging from room temperature to the boiling point of the reaction mixture, preferably at the boiling point of the reaction mixture);

(f) the compound of formula (8) is reacted with a malonate of the following formula (9):

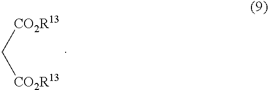

in which

R¹³ represents hydroxy or carboxy-protecting group, preferably t-butyldimethylsilyl, triisopropylsilyl, triethylsilyl, t-butyldiphenylsilyl, methoxymethyl, tetrahydropyranyl, methyl, ethyl, etc. in an inert solvent in the presence of a base to give a compound of the following formula (10):

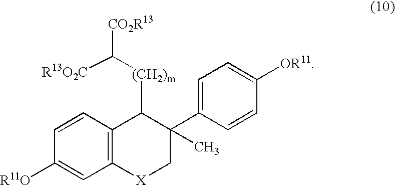

in which X, m, R¹¹ and R³ are defined as previously described (where tetrahydrofuran, dioxane, dichloromethane, dichloroethane, chloroform or dimethylsulfoxide, preferably tetrahydrofuran is used as the inert solvent, and sodium hydride, sodium hydroxide or potassium t-butoxide is used as the base, and the reaction is carried out at temperatures ranging from room temperature to the boiling point of the reaction mixture);

(g) the compound of formula (10) is reacted with a compound of the following formula (11):

CF₃CF₂(CH₃)ₙ-L² (11).

in which

n is defined as previously described, and

L² represents a leaving group, preferably methylsulfonyloxy, p-toluenesulfonyloxy, halogen, etc., in an inert solvent in the presence of a base to give a compound of the following formula (12):

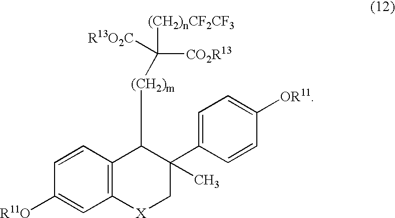

in which X, m, n, R¹¹ and R¹³ are defined as previously described (where the reaction conditions are the same as step (f));

(h) the compound of formula (12) is treated with sodium hydroxide or potassium hydroxide in an inert solvent to give a compound of the following formula (13):

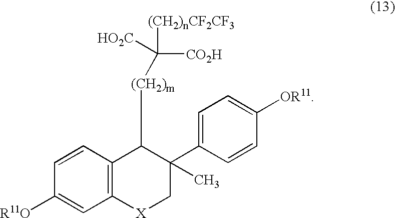

in which X, m, n and R¹¹ are defined as previously described (where water, ethanol, methanol, water-ethanol or water-methanol mixture is used as the inert solvent, and the reaction is carried out at temperatures ranging from room temperature to the boiling point of the reaction mixture, preferably at the boiling point of the reaction mixture);

(i) the compound of formula (13) is heated to a temperature of from 50° C. to the boiling point of the reaction mixture in an inert solvent and optionally in the presence of an acid to give a compound of the following formula (14):

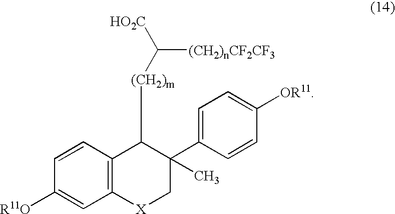

in which X, m, n and R¹¹ are defined as previously described (where dimethylsulfoxide, dimethylformamide, benzene, toluene, xylene, dioxane or tetrahydrofuran is used as the inert solvent, and hydrochloric acid, sulfuric acid or p-toluenesulfonic acid is used as the acid);

(j) the compound of formula (14) is deprotected by an acid to give a compound of the following formula (15):

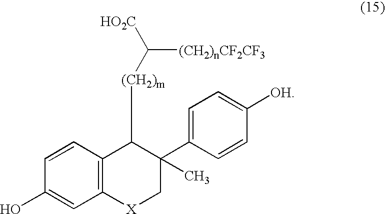

in which X, m, and n are defined as previously described (where hydrochloric acid, sulfuric acid, hydrobromic acid, hydrogen pyridinium chloride or borontribromide is used as the acid, and the reaction is carried out at temperatures ranging from −78° C. to the boiling point of the reaction mixture); and

(k) the compound of formula (15) is treated with a compound of the following formula (16):

R¹-L³ (16)

in which

R¹ is defined as previously described, and

L³ represents hydroxy, alkylcarbonyloxy, lower alkoxy, etc., in a solvent such as absolute methanol or ethanol to give the metal salt compound of formula (1).

(Process II)

The compound of formula (1) can also be prepared by a process characterized in that the compound of formula (13) obtained in step (h) of Process I is reacted according to the same procedure as step (j) to give a compound of the following formula (17):

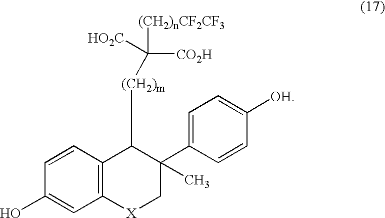

in which X, m and n are defined as previously described, the resulting compound (17) is reacted according to the same procedure as step (i) to give the compound of formula (15), which is then converted to a metal salt thereof according to the same procedure as step (k). That is, Process II produces the compound of formula (1) in the same manner as Process I except that the order of decarboxylation and deprotection of group R¹¹ is reversed. And the reaction conditions are same.

(Process III)

The compound of formula (1) can also be prepared by a process characterized in that the compound of formula (8) obtained in step (e) of Process I is reacted with a compound of the following formula (18):

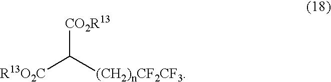

in which n and R¹³ are defined as previously described, in an inert solvent in the presence of a base to give the compound of formula (12) (where tetrahydrofuran, dioxane, dimethylsulfoxide, dichloromethane, dichloroethane or chloroform, preferably tetrahydrofuran is used as the inert solvent, and sodium hydride, sodium hydroxide or potassium t-butoxide is used as the base, and the reaction is carried out at temperatures ranging from −78° C. to the boiling point of the reaction mixture) and the following reactions are carried out according to the same procedure as Process I or II.

(Process IV)

The compound of formula (1) can also be prepared by a process characterized in that

(a) a compound of the following formula (19):

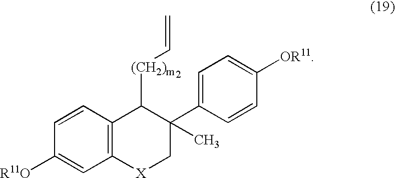

in which

X and R¹¹ are defined as previously described, and

m₂+m₃+2 equals m, is reacted with a compound of the following formula (20):

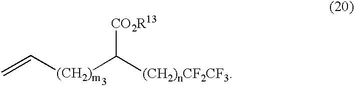

in which

n, m₃ and R¹³ are defined as previously described, in an inert solvent in the presence of a catalyst to give a compound of the following formula (21):

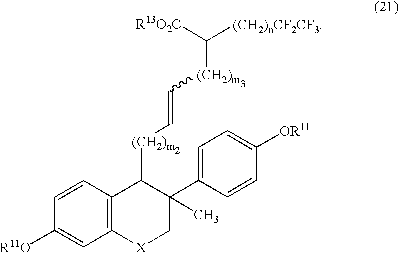

in which X, R¹¹, R¹³, n, m₂ and m₃ are defined as previously described (where dichloromethane, chloroform, bezene, toluene, xylene, dioxane, tetrahydrofuran, dimethylsulfoxide or dimethylformamide is used as the inert solvent, and benzylidene-bis(tricyclohexylphosphine)dichlororuthenium is used as the catalyst, and the reaction is carried out at temperatures ranging from −78° C. to the boiling point of the reaction mixture, preferably at the boiling point of the reaction mixture); and

(b) the compound of formula (21) is applied to a catalytic hydrogenation reaction in an inert solvent to give a compound of the following formula (22):

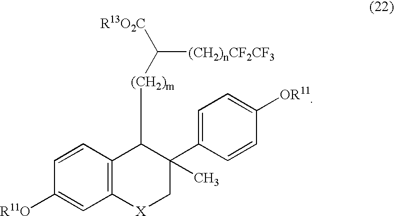

in which X, m, n, R¹¹ and R¹³ are defined as previously described (where methanol, ethanol, ethyl acetate, tetrahydrofuran, dioxane, dichloromethane, dichloroethane, chloroform or benzene is used as the inert solvent, and activated Pd/C, palladium hydroxide, platinum oxide or Wilkinson's catalyst is used as the catalyst, and the reaction is carried out at temperatures ranging from room temperature to the boiling point of the reaction mixture, preferably at room temperature)), and then hydrolysis, deprotection and conversion to the metal salt thereof are carried out according to the same procedure as Process I or II.

(Process V)

The compound of formula (1) can also be prepared by a process characterized in that

(a) the compound of formula (19) is reacted with a compound of the following formula (23):

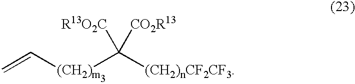

in which R¹³, n and m₃ are defined as previously described, in an inert solvent in the presence of a catalyst to give a compound of the following formula (24):

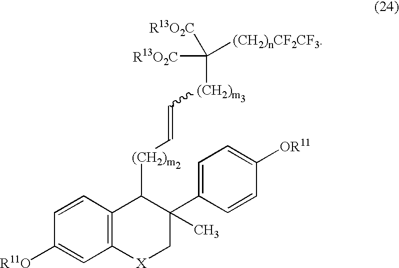

in which X, R¹¹, R¹³, n, m₂ and m₃ are defined as previously described (where the reaction conditions are the same as step (a) of Process IV); and

(b) the compound of formula (24) is applied to a catalytic hydrogenation reaction in a solvent to give a compound of the following formula (25):

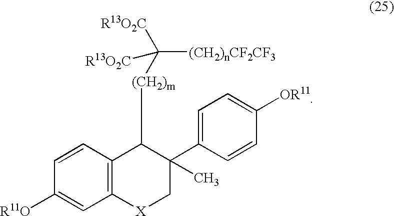

in which X, R¹¹, R¹³, n and m are defined as previously described (where the reaction conditions are the same as step (b) of Process IV), and then hydrolysis, decarboxylation, deprotection and conversion to the metal salt thereof are carried out according to the same procedure as Process I or II.

The compound of formula (1) thus prepared may be separated and purified using the conventional methods, such as for example, column chromatography, recrystallization, etc.

The above processes I to V according to the present invention will be more specifically explained through the following examples.

As stated above, the compound of formula (1) prepared according to the processes as explained above has a good anti-estrogenic activity and therefore, can be used for the treatment of estrogen-related diseases including anovular infertility, breast cancer, endometrial cancer, uterine cancer, ovarian cancer, endometriosis, endometrial fibroma, benign prostate hypertrophy, premature, menstrual disorder, etc.

Therefore, the present invention relates to an anti-estrogenic pharmaceutical composition comprising the compound of formula (1) as an active component together with pharmaceutically acceptable carriers.

When the anti-estrogenic pharmaceutical composition containing the compound of the present invention as an active component is used for clinical purpose, it can be formulated into a conventional preparation in the pharmaceutical field, for example, preparation for oral administration such as tablet, capsule, troche, solution, suspension, etc., or injectable preparation such as injectable solution or suspension, ready-to-use injectable dry powder which can be reconstituted with distilled water for injection when it is injected, etc., by combining with a carrier conventionally used in the pharmaceutical field.

Suitable carrier which can be used in the composition of the present invention includes those conventionally used in the pharmaceutical field, for example, binder, lubricant, disintegrant, excipient, solubilizer, dispersing agent, stabilizing agent, suspending agent, coloring agent, perfume, etc. for oral preparation; and preservative, pain alleviating agent, solubilizing agent, stabilizing agent, etc. for injectable preparation. The pharmaceutical preparation thus prepared can be administered orally or parenterally, for example, intravenously, subcutaneously or intraperitoneally. In addition, in order to prevent the active component from decomposition with gastric acid, the oral preparation can be administered together with an antacid or in the enteric-coated form of the solid preparation such as tablet.

The dosage of the metal salt of 3-methyl-chromane or thiocbromane derivative of formula (1) for human being can be suitably determined depending on absorption, inactivation and secretion of the active ingredient in the human body, age, sex and condition of subject patient, severity of the disease to be treated. It is generally suitable to administer the compound of formula (1) in an amount of 0.1 to 500 mg/day when it is orally administered, and in an amount of 1 to 1000 mg/month when it is parenterally administered (intravenous, intramuscular, or subcutaneous injection) for adult patient.

The present invention is more specifically explained by the following examples. However, it should be understood that the present invention is not limited to these examples in any manner.

### EXAMPLE 1

Synthesis of Sodium (3′RS,4′RS)-10-[7-hydroxy-3-(4-hydroxyphenyl)-3-methylthiochroman-4-yl]-2-(4,4,5,5,5-pentafluoropentyl)decanoate

(Step 1) 6-Bromo-1-(t-butyldimethylsilyloxy)hexane

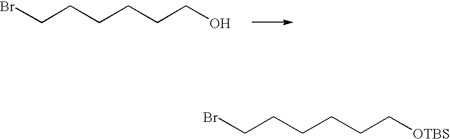

6-Bromohexan-1-ol (20 g, 110 mmol) was dissolved in anhydrous tetrahydrofuran (700 ml) and cooled to 0° C. under argon atmosphere, and then imidazole (15 g, 220 mmol) and t-butyldimethylsilyl chloride (33 g, 220 mmol) were added thereto. The reaction solution was stirred overnight. After the reaction was completed, the mixture was poured into ice-water and extracted with ethyl acetate. The organic solvent was dried over anhydrous magnesium sulfate and concentrated under reduced pressure. The crude product was purified by silica gel column chromatography (eluent: ethyl acetate/n-hexane=1/40, v/v) to give the title compound (30 g, Yield 92.4%) as a colorless oil.

¹H-NMR (300 MHz, CDCl₃) δ:3.61(t, 2H, J=6.4 Hz), 3.41(t, 2H, J=7.2 Hz), 1.90-1.73(m, 2H), 1.56-1.30(m, 6H), 0.89(s, 9H), 0.05(s, 6H)

(Step 2) 8-(t-Butyldimethylsilyloxy)-1-octyne

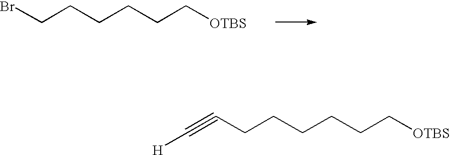

6-Bromo-1-(t-butyldimethylsilyloxy)hexane (20 g, 110 mmol) was dissolved in anhydrous dimethylsulfoxide (500 ml) and tetrahydrofuran (50 ml), and cooled to 0° C. under argon atmosphere. Lithium acetylide ethylenediamine complex (28.0 g, 304 mmol) was added thereto. The reaction solution was stirred for 1 day at 4° C. After the reaction was completed, the mixture was poured into ice-water and extracted with diethylether. The organic solvent was dried over anhydrous magnesium sulfate and concentrated under reduced pressure. The crude product was purified by silica gel column chromatography (eluent: ethyl acetate/n-hexane=1/40, v/v) to give the title compound (18 g, Yield 73.8%) as a colorless oil.

¹H-NMR (300 MHz, CDCl₃) δ: 3.59(t, 2H, J=6.4 Hz), 2.25-2.12(m, 2H), 1.90(t, 1H, J=2.4 Hz), 1.55˜1.30(m, 8H), 0.88(s, 9H), 0.05(s, 6H)

(Step 3) 4-[8-(t-Butyldimethylsilyloxy)-1-octynyl]-4-hydroxy-7-methoxy-3-(4-methoxyphenyl)-3-methylthiochromane

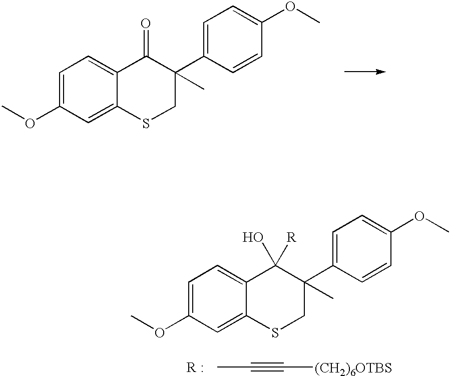

8-(t-Butyldimethylsilyloxy)-1-octyne (12 g, 50 mmol) was dissolved in anhydrous tetrahydrofuran (150 ml) under argon atmosphere and cooled to −78° C. 2.5M n-butyllithium (18 ml, 45 mmol) was added dropwise thereto. The mixture was warmed to −10° C., stirred for 1 hour, and then cooled to −78° C. again. 7-Methoxy-3-(4-methoxyphenyl)-3-methylthiochroman-4-one (7.84 g, 25 mmol) prepared according to the method known in WO98/25916 was added portionwise and the resulting mixture was warmed to room temperature and stirred for 1.5 hour. Water was added to the reaction mixture to stop the reaction. The reaction solvent was removed by evaporation and the residue was dissolved in ethyl acetate and then washed with water. The organic solvent was dried over anhydrous magnesium sulfate and removed by evaporation under vacuum. The crude product was purified by silica gel column chromatography (eluent: 10% ethyl acetate in n-hexane) to give the title compound (14 g, Yield 99.3%) as a colorless oil.

(Step 4) (3RS,4RS)-4-[8-(t-Butyldimethylsilyloxy)-1-octyl]-7-methoxy-3-(4-methoxyphenyl)-3-methylthiochromane

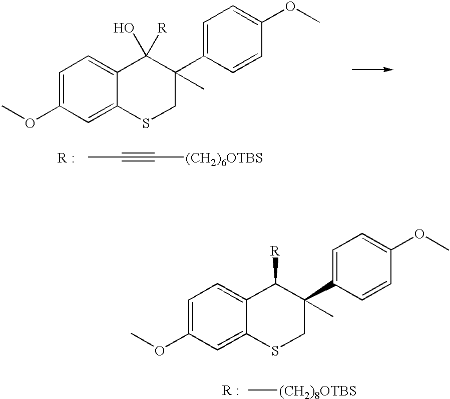

4-[8-(t-Butyldimethylsilyloxy)-1-octynyl]-4-hydroxy-7-methoxy-3-(4-methoxy phenyl)-3-methylthiochromane (14 g, 25 mmol) was dissolved in 1,2-dichloroethane (300 ml) and cooled to 0° C. Zinc(II) iodide (24.2 g, 75.7 mmol) and sodium cyanoborohydride (9.51 g, 151 mmol) were sequentially added thereto. The resulting mixture was slowly warmed to room temperature and stirred for 2 hours. After the reaction was completed, the reaction solvent was removed under reduced pressure. The residue was poured into water and extracted with ethyl acetate. The organic layer was dried over anhydrous magnesium sulfate and the solvent was removed by evaporation under vacuum. The product was purified by silica gel column chromatography (eluent: 10% ethyl acetate in n-bexane) to give a compound (11 g) as a pale yellow oil. Then, the compound (11 g) thus obtained was dissolved in tetrahydrofuran (300 ml). 0.2N sodium hydrogen carbonate (300 ml) and 10% Pd/C (3 g) were added and the mixture was stirred for 2 days under hydrogen atmosphere (normal pressure). The reaction mixture was filtered through cellite and concentrated under reduced pressure. The residue was dissolved in ethyl acetate and then washed with water and brine. The organic solvent was dried over anhydrous magnesium sulfate and removed by evaporation under vacuum. The crude product was purified by silica gel column chromatography (eluent: ethyl acetate/n-hexane=1/40, v/v) to give the title compound (5.6 g, Yield 50.5%) as a colorless oil.

¹H NMR (300 MHz, CDCl₃) δ: 7.27(d, 2H, J=9.0 Hz), 6.92-6.86(m, 3H), 6.70(d, 1H, J=2.6 Hz), 6.56(dd, 1H, J=8.7, 2.6 Hz), 3.79(s, 3H), 3.76(s, 3H), 3.62(d, 1H, J=11.7 Hz), 3.53(t, 2H, J=6.4 Hz), 2.96(d, 1H, J=12.7 Hz), 2.70(m, 1H), 1.46-1.37(m, 2H), 1.15(s, 3H), 1.20-0.90(m, 12H), 0.86(s, 9H), 0.01(s, 6H)

(Step 5) (3RS,4RS)-4-(8-Hydroxyoctyl)-7-methoxy-3-(4-methoxyphenyl)-3-methylthiochromane

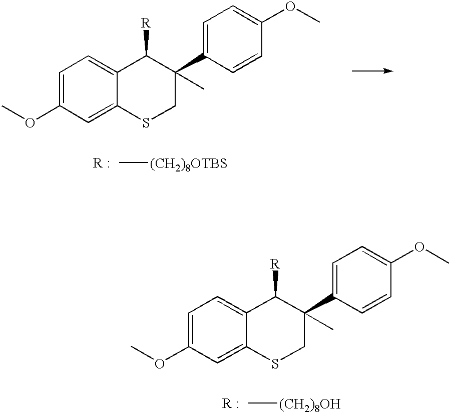

(3RS,4RS)-4-[8-(t-Butyldimethylsilyloxy)-1-octyl]-7-methoxy-3-(4-methoxy phenyl)-3-methylthiochromane (4.5 g, 8.3 mmol) was dissolved in tetrahydrofuran (100 ml) and cooled to 0° C. To this solution was added tetrabutylammonium fluoride (16.6 ml, 16.6 mmol) and the reaction mixture was stirred for 2 hours at room temperature. The solvent was removed by evaporation under reduced pressure and the resulting residue was dissolved in ethyl acetate and washed with water. The organic layer was separated, dried over anhydrous magnesium sulfate and concentrated under reduced pressure. The product was purified by silica gel column chromatography (eluent: 30% ethyl acetate in n-hexane) to give the title compound (3.3 g, Yield 93.0%) as a pale yellow oil.

¹H-NMR (300 MHz, CDCl₃, 3RS,4RS-compound) δ: 7.27(d, 2H, J=8.7 Hz), 6.92-6.87(m, 3H), 6.70(d, 1H, J=2.6 Hz), 6.56(dd, 1H, J=8.7, 2.6 Hz), 3.80(s, 3H), 3.76(s, 3H), 3.67(d, 1H, J=13.2 Hz), 3.53(t, 2H, J=6.4 Hz), 2.96(d, 1H, J=11.7 Hz), 2.71(m, 1H), 1.16(s, 3H), 1.30-1.07(m, 14H)

(Step 6) (3RS,4RS)-4-(8-Methylsulfonyloxyoctyl)-7-methoxy-3-(4-methoxy phenyl)-3-methylthiochromane

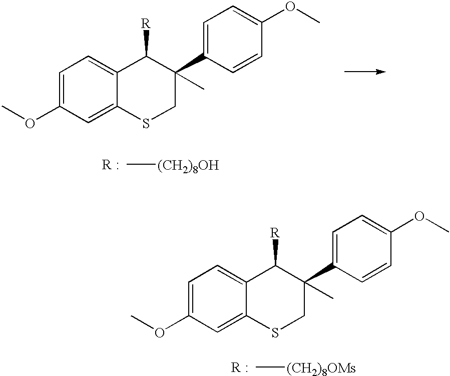

(3RS,4RS)-4-(8-hydroxyoctyl)-7-methoxy-3-(4-methoxyphenyl)-3-methylthiochroman (3.25 g, 7.6 mmol) was dissolved in dichloromethane (100 ml), and then triethylamine (1.59 ml, 11.4 mmol) and methylsulfonylchloride (0.88 ml, 11.4 mmol) were added thereto. The reaction mixture was stirred for 1 hour at room temperature. After the reaction was completed, water was added to the reaction solution and the resulting mixture was extracted with methylene chloride. The organic layer was washed with 1M hydrochloric acid solution, water and saturated sodium chloride solution, dried over anhydrous magnesium sulfate, and concentrated. The product was purified by silica gel column chromatography (eluent: 30% ethyl acetate in n-hexane) to give the title compound (3.75 g, Yield 97.7%) as a pale yellow oil.

¹H-NMR (300 MHz, CDCl₃) δ: 7.27(d, 2H, J=9.0 Hz), 6.92-6.87(m, 3H), 6.70(d, 1H, J=2.6 Hz), 6.56(dd, 1H, J=8.3, 2.6 Hz), 4.15(t, 2H, J=6.4 Hz), 3.80(s, 3H), 3.76(s, 3H), 3.62(d, 1H, J=11.7 Hz), 2.97(d, 1H, J=11.7 Hz), 2.96(s, 3H), 2.71(m, 1H), 1.69-1.59(m, 2H), 1.15(s, 3H), 1.20˜0.96(m, 12H)

(Step 7) (3RS,4RS)-4-(8-Iodooctyl)-7-methoxy-3-(4-methoxyphenyl)-3-methylthiochromane

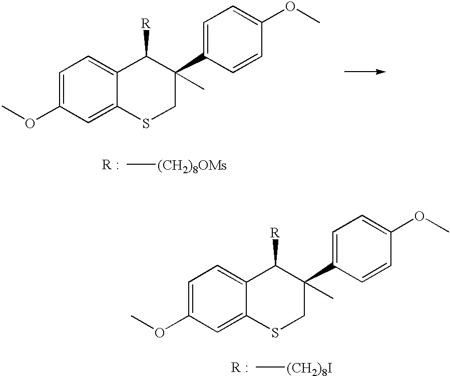

(3RS,4RS)-4-(8-methylsulfonyloxyoctyl)-7-methoxy-3-(4-methoxyphenyl)-3-methylthiochromane (3.75 g, 7.4 mmol) was dissolved in acetone (70%), to which was added sodium iodide (3.33 g, 22.2 mmol). The reaction mixture was heated to reflux temperature for 4 hours while stirring, cooled to room temperature, and then concentrated under reduced pressure to remove acetone. The residue was dissolved in ethyl acetate and then filtered. The organic layer was washed with 1% sodium thiosulfate solution, water and brine, and then dried over anhydrous magnesium sulfate. The solvent was removed by evaporation under vacuum and the resulting product was purified by silica gel column chromatography (eluent: 10% ethyl acetate in n-hexane) to give the title compound (3.73 g, Yield 93.6%) as a colorless oil.

¹H-NMR (300 MHz, CDCl₃, 3RS,4RS-compound) δ: 7.27(d, 2H, J=8.7 Hz), 6.92-6.86(m, 3H), 6.72(d, 1H, J=2.6 Hz), 6.56(dd, 1H, J=8.7, 2.6 Hz), 3.81(s, 3H), 3.76(s, 3H), 3.63(d, 1H, J=11.7 Hz), 3.12(t, 2H, J=6.8 Hz), 2.96(d, 1H, J=11.7 Hz), 2.70(m, 1H), 1.76-1.68(m, 2H), 1.16(s, 3H), 1.28˜1.00(m, 12H)

(Step 8) 1-Methylsulfonyloxy-4,4,5,5,5-pentafluoropentane

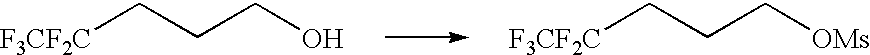

4,4,5,5,5-pentafluoropentan-1-ol (25 g, 0.13 mol) was dissolved in dichloromethane (50 ml) and cooled to 0° C., and then triethylamine (46 ml, 0.33 mol) and methylsulfonylchloride (20.4 ml, 0.26 mol) were added thereto. The reaction mixture was stirred for 3 hours at room temperature. After the reaction was completed, water was added to the reaction solution and the resulting mixture was extracted with dichloromethane. The organic layer was washed with 1M hydrochloric acid solution, water and saturated sodium chloride solution, dried over anhydrous magnesium sulfate, and concentrated. The product was purified by silica gel column chromatography (eluent: 50% ethyl acetate in n-hexane) to give the title compound (34 g, Yield: quantitative) as a pale yellow oil.

¹H-NMR (300 MHz, CDCl₃) δ: 4.30(t, 2H), 3.05(s, 3H), 2.30˜2.05(m, 4H)

(Step 9) 1-Iodo-4,4,5,5,5-pentafluoropentane

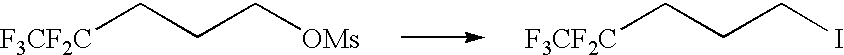

1-Methylsulfonyloxy-4,4,5,5,5-pentafluoropentane (20 g, 0.8 mol) was dissolved in acetone (200 ml), and sodium iodide (35.1 g, 2.2 mol) was added to the reaction solution. The reaction mixture was stirred at reflux temperature overnight, cooled to room temperature, filtered, and concentrated under reduced pressure. The residue was dissolved in diethylether and filtered. The organic layer was washed with water and brine, dried over anhydrous magnesium sulfate, and evaporated under reduced pressure to remove the solvent and to give the title compound (21.1 g, Yield 93.4%) as a colorless oil.

¹H-NMR (300 MHz, CDCl₃) δ: 3.22(t, 2H, J=6.8 Hz), 2.28˜2.10(m, 4H)

(Step 10) Diethyl 2-(4,4,5,5,5-pentafluoropentyl)propan-1,3-dioate

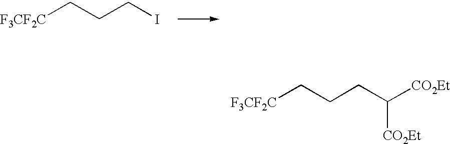

To a solution of sodium hydride (60%)(3.90 g, 96.07 mol) in tetrahydrofuran (160 ml) was added dropwise diethyl malonate solution (16.6 ml, 110.85 mol) under ice-cooling and the resulting mixture was stirred for 30 minutes. Then, 1-iodo-4,4,5,5,5-pentafluoropentane (21 g, 73.9 mol) was added dropwise thereto, and the reaction mixture was warmed to room temperature overnight while stirring. After the reaction was completed, water was added to stop the reaction, and the reaction solution was extracted with ethyl acetate. The organic layer was washed with water and brine, dried over anhydrous magnesium sulfate, and evaporated under reduced pressure to remove the solvent. The product was purified by silica gel column chromatography (eluent: dichloromethane/n-hexane=1/3,v/v) to give the title compound (18.3 g, Yield 78.4%) as a pale yellow oil.

¹H-NMR (300 MHz, CDCl₃) δ: 4.22(q, 4H, J=7.1 Hz), 3.35(t, 1H, J=6.0 Hz), 2.15˜1.93(m, 4H), 1.73-1.55(m, 2H), 1.28(t, 6H, J=7.11 Hz)

(Step 11) (3′RS,4′RS)-Diethyl 2-{8-[7-methoxy-3-(4-methoxyphenyl)-3-methylthiochroman-4-yl]octyl}-2-(4,4,5,5,5-pentafluoropentyl)propan-1,3-dioate

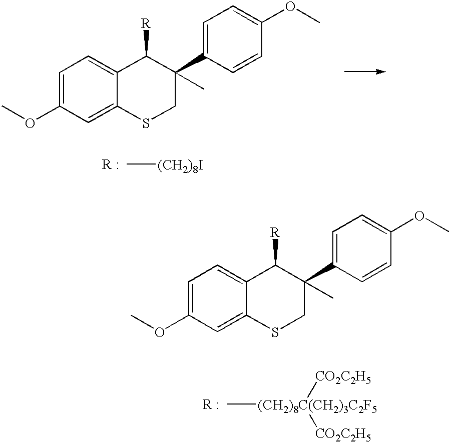

To a solution of sodium hydride (60%)(312 mg, 7.8 mmol) in tetrahydrofuran (14 ml) was added dropwise a solution of diethyl 2-(4,4,5,5,5-pentafluoropentyl)propan-1,3-dioate (2.38 g, 7.4 mmol) in tetrahydrofuran (8 ml) under ice-cooling, which was then stirred for 1 hour. Then, a solution of (3RS,4RS)-4-(8-iodooctyl)-7-methoxy-3-(4-methoxy-phenyl)-3-methylthiochromane (2.0 g, 3.7 mmol) in tetrahydrofuran (8 ml) was added dropwise thereto, and the reaction mixture was warmed to room temperature for 2 days while stirring. After the reaction was completed, water was added to stop the reaction and the mixture was extracted with ethyl acetate. The organic layer was washed with water and brine, dried over anhydrous magnesium sulfate, and evaporated under vacuum to remove the solvent. The product was purified by silica gel column chromatography (eluent: ethyl acetate/n-hexane=1/4→1/1, v/v) to give the title compound (2.60 g, Yield 95.9%) as a colorless oil.

¹H-NMR (300 MHz, CDCl₃) δ: 7.26(d, 2H, J=8.7 Hz), 6.92-6.86(m, 3H), 6.70(d, 1H, J=2.6 Hz), 6.56(dd, 1H, J=8.3, 2.6 Hz), 4.15(q, 4H, J=7.1 Hz), 3.80(s, 3H), 3.76(s, 3H), 3.62(d, 1H, J=11.7 Hz), 2.96(d, 1H, J=11.7 Hz), 2.70(m, 1H), 2.05-1.78(m, 6H), 1.48-1.38(m, 2H), 1.15(s, 3H), 1.20-0.96(m, 20H)

(Step 12) (3RS,4′RS)-{8-[7-methoxy-3-(4-methoxyphenyl)-3-methylthiochroman-4-yl]octyl}(4,4,5,5,5-pentafluoropentyl)methane-1,1-dicarboxylic Acid

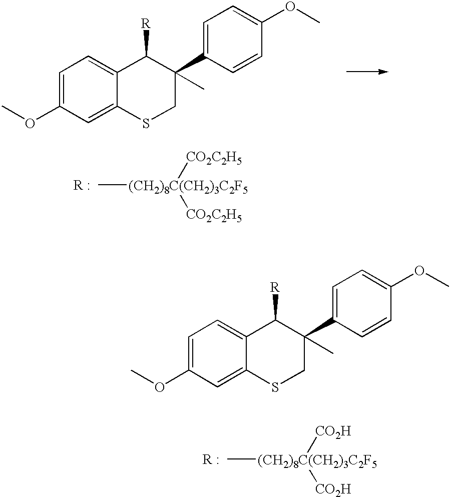

To a solution of (3′RS,4′RS)-diethyl 2-{8-[7-methoxy-3-(4-methoxyphenyl)-3-methylthiochroman-4-yl]octyl}-2-(4,4,5,5,5-pentafluoropentyl)propan-1,3-dioate (2.6 g, 3.56 mmol) in ethyl alcohol (40 ml) was added aqueous potassium hydroxide (7.8 g, 142.3 mmol) solution (20 ml). The mixture was reflux-heated overnight. The residue which was obtained by removing ethyl alcohol was dissolved in water and the mixture was extracted with ethyl acetate. The organic layer was washed with water and brine, dried over anhydrous magnesium sulfate, and evaporated under vacuum to remove the solvent and to give the title compound (2.30 g, Yield: quantitative) as a white foam.

¹H-NMR (300 Hz, CDCl₃) δ: 7.28(d, 2H, J=8.7 Hz), 6.93-6.86(m, 3H), 6.71(d, 1H, J=2.6 Hz), 6.57(dd, 1H, J=8.3, 2.6 Hz), 3.82(s, 3H), 3.77(s, 3H), 3.62(d, 1H, J=111.7 Hz), 2.97(d, 1H, J=11.7 Hz), 2.70(m, 1H), 2.10-1.82(m, 6H), 1.60-1.47(m, 2H), 1.15(s, 3H), 1.13˜1.00(m, 14H)

(Step 13) (3′RS,4′RS)-10-[7-methoxy-3-(4-methoxyphenyl)-3-methylthiochroman-4-yl]-2-(4,4,5,5,5-pentafluoropentyl)decanoic Acid

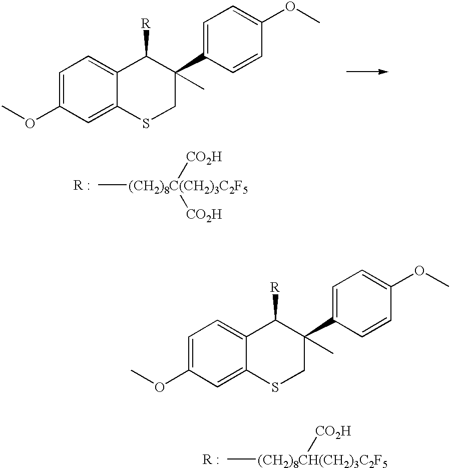

In dimethylsulfoxide (20 ml) was dissolved (3′RS,4′RS)-{8-[7-methoxy-3-(4-methoxyphenyl)-3-methylthiochroman-4-yl]octyl}(4,4,5,5,5-pentafluoropentyl)methane-1,1-dicarboxylic acid (2.0 g, 3.1 mol), which was then heated to 130˜140° C. for 4 hours. The reaction mixture was dissolved in ethyl acetate (300 ml), washed with water (20 ml×4) and brine (20 ml×2), and dried over anhydrous magnesium sulfate. The product was purified by silica gel column chromatography (eluent: ethyl acetate/n-hexane=1/9→1/5→1/1, v/v) to give the title compound (1.60 g, Yield 82.1%) as a white foam.

¹H-NMR (300 MHz, CDCl₃) δ: 7.27(d, 2H, J=8.7 Hz), 6.92-6.87(m, 3H), 6.70(d, 1H, J=2.6 Hz), 6.56(dd, 1H, J=8.3, 2.6 Hz), 3.80(s, 3H), 3.76(s, 3H), 3.62(d, 1H, J=11.7 Hz), 2.96(d, 1H, J=11.7 Hz), 2.70(m, 1H), 2.34(m, 1H), 2.06-1.90(m, 2H), 1.70˜1.40(m, 6H), 1.15(s, 3H), 1.20˜10.00(m, 14H)

(Step 14) (3′RS,4′RS)-10-[7-Hydroxy-3-(4-hydroxyphenyl)-3-methylthiochroman-4-yl]-2-(4,4,5,5,5-pentafluoropentyl)decanoic Acid

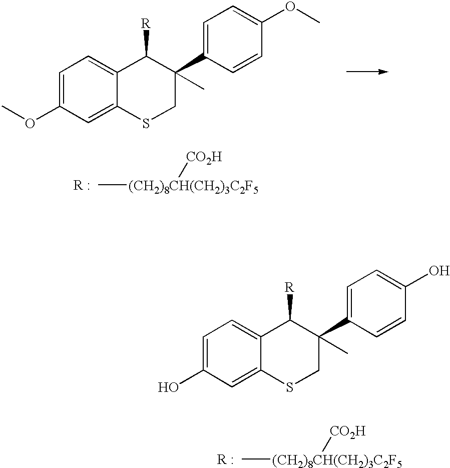

A solution of (3′RS,4′RS)-10-[7-methoxy-3-(4-methoxyphenyl)-3-methylthiochroman-4-yl]-2-(4,4,5,5,5-pentafluoropentyl)decanoic acid in anhydrous dichloromethane (30 ml) was cooled to −78° C. under argon atmosphere. To this solution was added dropwise borontribromide (1.0 mol/l dichloromethane solution, 12.4 ml, 12.4 mmol) for 10 minutes and the resulting solution was stirred for 1 hour, warmed to −5° C., and stirred for 1 hour. After the reaction was completed, the mixture was poured into ice water and extracted with dichloromethane. The organic solvent was dried over anhydrous magnesium sulfate and concentrated under vacuum. The crude product was purified by silica gel column chromatography (eluent: ethyl acetate/n-hexane=1/5→1/3→2/3, v/v) to give the title compound (1.1 g, Yield 88.7%) as a white foam.

¹H-NMR (300 MHz, CDCl₃) δ: 7.16(d, 2H, J=8.7 Hz), 6.90-6.78(m, 3H), 6.60(d, 1H, J=2.4 Hz), 6.56(dd, 1H, J=8.2, 2.4 Hz), 3.55(d, 1H, J=11.5 Hz), 2.88(d, 1H, J=11.5 Hz), 2.62(m, 1H), 2.33(m, 1H), 2.10-1.9(m, 2H), 1.70˜1.40(m, 6H), 1.10(s, 3H), 1.10˜1.00(m, 14H)

Mass (ESI): 625[M+Na], 603[M+1]

(Step 15) Sodium (3′RS,4′RS)-10-[7-hydroxy-3-(4-hydroxyphenyl)-3-methylthiochroman-4-yl]-2-(4,4,5,5,5-pentafluoropentyl)decanoate

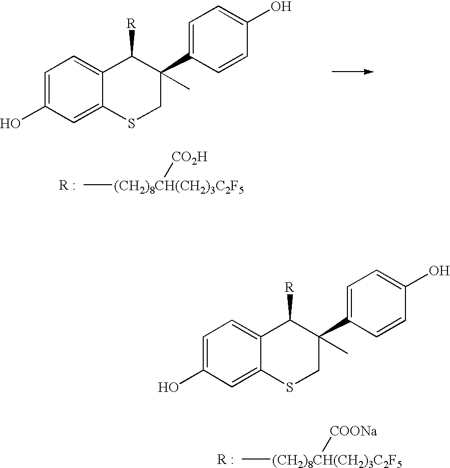

To a solution of (3′RS,4′RS)-10-[7-hydroxy-3-(4-hydroxyphenyl)-3-methylthiochroman-4-yl]-2-(4,4,5,5,5-pentafluoropentyl)decanoic acid (458 mg, 0.753 mmol) in absolute methanol (2 ml) was added sodium methoxide (0.75 ml, 0.75 mmol) at room temperature, which was then stirred for 1.5 hour at the same temperature. Then, anhydrous diethylether was added to the reaction mixture and evaporated under reduced pressure to give the title compound (454 mg, Yield 95.6%) as a white amorphous solid.

¹H-NMR (270 MHz, CD₃OD): δ 7.17(d, J=8.9 Hz, 2H, Ar—H), 6.79(d, J=8.2 Hz, 1H, Ar—H), 6.73(d, J=8.3 Hz, 2H, Ar—H), 6.49(d, J=2.7 Hz, 1H, C8-H), 6.38(dd, J=8.3 Hz and 2.7 Hz, 1H, C6-H), 3.56(d, J=11.5 Hz, 1H, C2-H), 2.88(d, J=11.5 Hz, 1H, C2-H), 2.66(m, 1H, C4-H), 2.02(m, 3H, alkyl-H), 1.54-1.07(m, 23H, C₃-CH₃ and alkyl-H)

### EXAMPLE 2

Synthesis of Sodium (3′RS,4′RS)-11-[7-hydroxy-3-(4-hydroxyphenyl)-3-methylthiochroman-4-yl]-2-(4,4,5,5,5-pentafluoropentyl)undecanoate

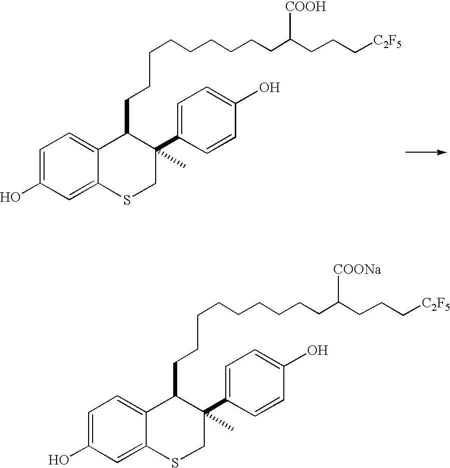

(3′RS,4′RS)-11-[7-Hydroxy-3-(4-hydroxyphenyl)-3-methylthiochroman-4-yl]-2-(4,4,5,5,5-pentafluoropentyl)undecanoic acid (119 mg, 0.193 mmol) prepared according to a similar procedure as Steps 1 to 14 of Example 1 was added to ethanol (2 ml) under argon atmosphere, sodium ethoxide (62.5 μg, 0.193 mmol) was added thereto, and the resulting mixture was stirred for 3 hours at room temperature. The solvent was removed under reduced pressure to give the title compound (0.123 mg, Yield 100%) as a gray foam.

¹H-NMR(300 MHz, CDCl₃): δ 7.22(d, J=9.1 Hz, 2H), 6.83(d, J=8.3 Hz, 1H), 6.77(d, J=8.7 Hz, 2H), 6.54(d, J=2.3 Hz, 1H), 6.43(dd, J=8.40, 2.36 Hz, 1H), 3.60(d, J=12.1 Hz, 1H), 2.93(d, J=11.4 Hz, 1H), 2.72(m, 1H), 2.19-2.02(m, 3H), 1.64-0.95(m, 25H)

### EXAMPLE 3

Synthesis of Sodium (3′RS,4′RS)-11-[7-hydroxy-3-(4-hydroxyphenyl)-3-methylchroman-4-yl]-2-(4,4,5,5,5-pentafluoropentyl)undecanoate

(Step 1) 4,4,5,5,5-Pentafluoropentyl Iodide

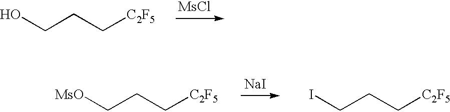

4,4,5,5,5-Pentafluoropentan-1-ol (100 g, 0.56 mol) and triethylamine (200 ml) were added to dichloromethane (2,000 ml) under argon atmosphere and cooled to 0° C. Methylsulfonylchloride (52 ml, 0.67 mol) was slowly added dropwise thereto and stirred for 1.5 hour. Ice which had been broken into pieces was added to stop the reaction, and the organic layer was washed with water and saturated saline solution and dried over anhydrous magnesium sulfate. The filtrate was concentrated under reduced pressure and the residue was purified by column chromatography (eluent: n-hexane/ethyl acetate=4/1, v/v) to give 1-methylsulfonyloxy-4,4,5,5,5-pentafluoropentane (160 g, quantitative) as a yellow oil.

1-Methylsulfonyloxy-4,4,5,5,5-pentafluoropentane (160 g, 0.63 mol) and sodium iodide (280 g, 1.88 mol) were added to acetone (3,000 ml) and refluxed overnight. After the mixture was cooled to room temperature, water (5,000 ml) was added. The resulting mixture was extracted with diethylether. The organic layer was washed with water and saturated saline solution, dried over anhydrous magnesium sulfate, filtered and concentrated under reduced pressure to give the title compound (108 g, Yield 67%) as a yellow oil.

¹H-NMR(300 MHz, CDCl₃): δ 3.23 (t, 2H), 2.26-2.12 (m, 4H)

(Step 2) Diethyl 2-oct-7-enyl-2-(4,4,5,5,5-pentafluoropentyl)propan-1,3-dioate

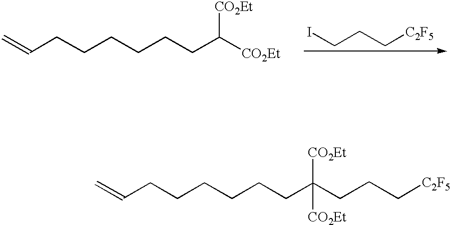

Diethyl 2-oct-7-enylpropan-1,3-dioate (19 g, 70.3 mmol) was added to tetrahydrofuran (200 ml) under argon atmosphere and cooled to 0° C. After sodium hydride (5.62 g, 140.6 mmol) was slowly added, 4,4,5,5,5-pentafluoropentyl iodide (40.5 g, 140.6 mmol) was added, and the mixture was refluxed for 2 hours. The reaction mixture was cooled to room temperature and water was added thereto. The organic layer obtained by extraction with ethyl acetate was washed with water and saturated saline solution, dried over anhydrous magnesium sulfate, filtered and concentrated under reduced pressure. The residue was purified by column chromatography (eluent: n-hexane/ethyl acetate=50/1, v/v) to give the title compound (25.6 g, Yield 85%) as a colorless oil.

¹H-NMR(300 MHz, CDCl₃): δ 5.77 (m, 1H), 5.01-4.92 (m, 2H), 4.19 (q, 4H, J=6.8 Hz), 2.08-1.86 (m, 8H), 1.57-1.15 (m, 16H)

(Step 3) Ethyl 2-(4,4,5,5,5-pentafluoropentyl)-9-decenoate

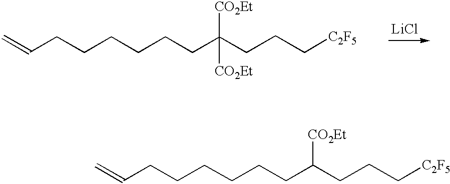

Diethyl 2-oct-7-enyl-2-(4,4,5,5,5-pentafluoropentyl)propan-1,3-dioate (16.6 g, 38.5 mmol), lithium chloride (3.24 g, 77.0 mmol) and water (0.7 ml, 38.5 mmol) were added to DMSO (200 ml), which was then stirred for 12 hours at 170° C. After the mixture was cooled to room temperature, the organic layer obtained by extraction with ethyl acetate was washed with water and saturated saline solution, dried over anhydrous magnesium sulfate, filtered and concentrated under reduced pressure. The residue was purified by column chromatography (eluent: n-hexane/ethyl acetate=30/1, v/v) to give the title compound (10 g, Yield 73%) as a colorless oil.

¹H-NMR(300 MHz, CDCl₃): δ 5.80 (m, 1H), 4.97 (m, 2H), 4.15 (q, 2H, J=7.1 Hz), 2.34 (m, 1H), 1.99-2.04 (m, 4H), 1.75-1.26 (m, 20H)

(Step 4) 7-Methoxymethoxy-3-(4-methoxymethoxyphenyl)-4-(2-propenyl) chroman-2-one

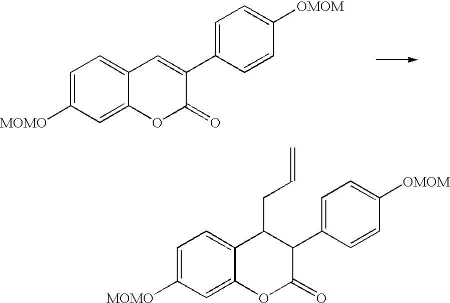

To tetrabutylammoniumfluoride hydrate (6 g) were added 20 ml each of toluene and ethanol. After the mixture was dehydrated under reduced pressure, toluene was added in an amount of 20 ml per each time and dehydrated under reduced pressure twice. The oily substance having a light yellow color thus obtained was dried under vacuum pump to give anhydrous tetrabutylammoniumfluoride. This anhydrous tetrabutylammoniumfluoride-containing anhydrous dimethylformamide solution (80 ml) was added to a suspension of 7-methoxymethoxy-3-(4-methoxymethoxyphenyl)-chromen-2-one (14.8 g) in anhydrous dimethylformamide (80 ml). To this suspension was added dropwise a solution of HMPA (distillated under reduced pressure in the presence of calcium hydride, 27.1 ml) and allyltrimethylsilane (24.7 ml) in anhydrous dimethylformamide (80 ml) for 15 minutes at room temperature. The reaction mixture having a red color was stirred for 2 hours at room temperature, and then the reaction was stopped by the addition of methanol (200 ml) solution containing 1N hydrochloric acid (100 ml) in ice-water. The mixture was extracted three times with ethyl acetate, and the organic layer was washed three times with water and dried over anhydrous magnesium sulfate. The crude product obtained by concentration under reduced pressure was purified by column chromatography (eluent: n-hexane/ethyl acetate=10/1→9/1, v/v) to give the title compound (14.1 g, Yield 85.0%) as a yellow oil.

¹H-NMR(270 MHz, CDCl₃): δ 7.23(d, 1H, J=8.6 Hz, Ar—H), 7.0-7.1(m, 3H, Ar—H), 6.90(d, 1H, J=8.6 Hz, Ar—H), 6.7-6.9(m, 2H, Ar—H), 5.5-5.9(m, 1H, vinyl-H), 5.10, 5.15, 5.18(each s, 4H, OCH₂OMe), 4.8-5.2(m, 2H, vinyl-H), 4.14(d, 0.4H, J=5.6 Hz, C3-H), 4.03(d, 0.6H, J=3.3 Hz, C3-H), 3.50, 3.48, 3.43(each s, 6H, OCH₃), 3.19(td, 0.6H, J=6.9, 3.3 Hz, C4-H), 3.05-3.15(m, 0.4H, C4-H), 2.1-2.5(m, 2H, allyl-H)

(Step 5) (3RS,4RS)-7-Methoxymethoxy-3-(4-methoxymethoxyphenyl)-3-methyl-4-(2-propenyl)chroman-2-one

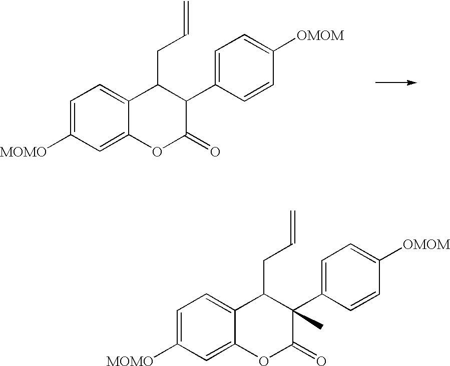

To a solution of 7-methoxymethoxy-3-(4-methoxymethoxyphenyl)-4-(2-propenyl)chroman-2-one (32.70 g) in anhydrous tetrahydrofuran (400 ml) was added dropwise 1M solution of lithium hexamethyldisilazide in tetrahydrofuran (170 ml) for 15 minutes at −73° C. under nitrogen atmosphere, which was then stirred for 30 minutes at −10° C. The reaction mixture was cooled to −75° C., methyliodide (10.6 ml) was added dropwise thereto for 10 minutes, and then stirred for 10 minutes at −75° C., 1 hour at −10° C., and 1 hour at 0° C. The reaction was stopped by the addition of saturated aqueous ammonium chloride solution, and then ethyl acetate and water were added thereto. The organic layer was washed twice with water, dried over anhydrous sodium sulfate and concentrated under reduced pressure. The residue was purified by column chromatography (eluent: n-hexane/ethyl acetate=9/1, v/v) to give the title compound (30.74 g, Yield 90.8%) as a yellow oil.

¹H-NMR(270 MHz, CDCl₃): δ 7.44(d, 2H, J=8.6 Hz, Ar—H), 7.00-7.05(m, 3H, Ar—H), 6.75-6.80(m, 2H, Ar—H), 5.40-5.65(m, 1H, vinyl-H), 5.18, 5.17(each s, 4H, OCH₂Me), 4.92(d, 1H, J=10.2 Hz, vinyl-H), 4.79(d, 1H, J=17.2 Hz, vinyl-H), 3.50, 3.49(each s, 6H, OCH₃), 2.84(dd, 1H, J=9.9, 3 Hz, C4-H), 2.15-2.30(m, 1H, allyl-H), 1.90-2.05(m, 1H, allyl-H), 1.61(s, 3H, C₃-CH₃)

(Step 6) (2RS,3RS)-3-(2-Hydroxy-4-methoxymethoxyphenyl)-2-(4-methoxymethoxyphenyl)-2-methyl-5-hexen-1-ol

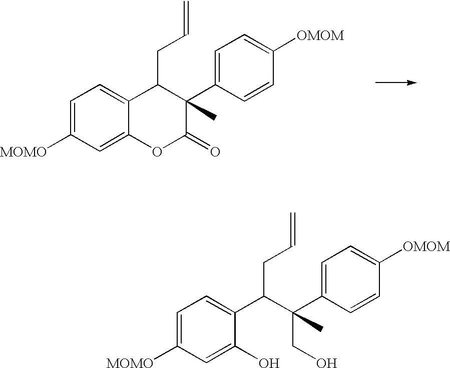

A anhydrous tetrahydrofuran solution (26 ml) containing (3RS,4RS)-7-methoxymethoxy-3-(4-methoxymethoxyphenyl)-3-methyl-4-(2-propenyl)chroman-2-one (6.91 g) was added dropwise to a anhydrous tetrahydrofuran suspension (50 ml) containing lithium aluminum hydride (1.65 g) which had been cooled by ice-water for 20 minutes under nitrogen atmosphere, which was then stirred for 50 minutes under ice-water. Ethyl acetate (20 ml) and saturated aqueous ammonium chloride solution (20 ml) were added to stop the reaction and stirred for 1 hour at room temperature. The reaction mixture was filtered through cellite and the filtrate was extracted twice with ethyl acetate. The organic layer was washed with saturated aqueous ammonium chloride solution, dried over anhydrous magnesium sulfate and concentrated under reduced pressure to give the title compound (6.94 g, Yield 99.4%) as a crude product which was directly used in the next reaction.

¹H-NMR(270 MHz, CDCl₃): δ 7.3-7.6(bs, 1H, Ar—OH), 6.9-7.1(m, 4H, Ar—H), 6.57(d, 1H, J=2.3 Hz, Ar—H), 6.37(dd, 1H, J=8.6, 2.3 Hz, Ar—H), 6.05-6.25(d, 1H, Ar—H), 6.3-6.5(m, 1H, vinyl-H), 5.18(s, 2H, OCH₂OMe), 5.12(d, 2H, J=6.9 Hz, OCH₂OMe), 4.84(d, 1H, J=17.2 Hz, vinyl-H), 4.75(d, 1H, J=10.2 Hz, vinyl-H), 3.92(d, 1H, J=9.9 Hz, C1-H), 3.49, 3.50(each s, 6H, OCH₃), 3.4-3.5(m, 1H, C1-H), 3.25-3.35(m, 1H, C3-H), 2.8-3.0(d, 1H, OH), 2.5-2.7(m, 1H, C4-H), 1.95-2.2(m, 1H, C4-H), 1.63(s, 3H, C₃-CH₃)

(Step 7) (3RS,4RS)-7-Methoxymethoxy-3-(4-methoxymethoxyphenyl)-3-methyl-4-(2-propenyl)chromane

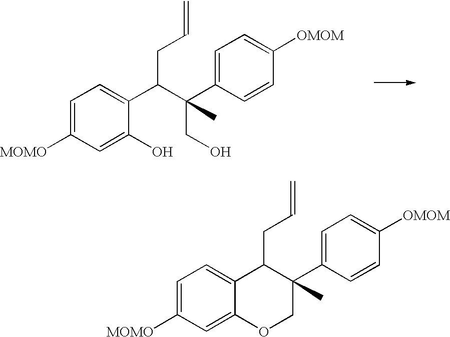

A solution of (2RS,3RS)-3-(2-hydroxy-4-methoxymethoxyphenyl)-2-(4-methoxymethoxyphenyl)-2-methyl-5-hexen-1-ol (6.9 g) and triphenylphosphine (1.24 g) in anhydrous 1,4-dioxane (120 ml) was cooled in ice-water under nitrogen atmosphere. Diethylazodicarboxylate (5.94 ml) was added dropwise for 25 minutes and stirred for 20 minutes at room temperature. Water was added to the reaction mixture, which was then extracted twice with ethyl acetate. The organic layer was dried over anhydrous magnesium sulfate and concentrated under reduced pressure. The crude product was purified by flash column chromatography (eluent: n-hexane/ethyl acetate=100/1→20/1→9/1, v/v) to give the title compound (6.5 g, Yield 98.6%) as a colorless oil.

¹H-NMR(270 MHz, CDCl₃): δ 7.03, 7.15(each d, 4H, J=8.9 Hz, Ar—H), 6.96 (d, 1H, J=7.9 Hz, Ar—H), 6.5-6.6(m, 2H, Ar—H), 5.5-5.7(m, 1H, vinyl-H), 5.18(s, 2H, OCH₂OMe), 5.14(d, 2H, J=6.9 Hz, OCH₂OMe), 4.86(dd, 1H, J=10.2, 1.3 Hz, vinyl-H), 4.70(dd, 1H, J=17.2, 1.3 Hz, vinyl-H), 4.52(d, 1H, J=10.2 Hz, C2-H), 4.26(dd, 1H, J=10.6, 2.0 Hz, C2-H), 3.49, 3.50(each s, 6H, OCH₃), 3.75-3.85(m, 1H, C4-H), 2.0-2.1(m, 1H, allyl-H), 1.8-1.9(m, 1H, allyl-H), 1.29 (s, 3H, C₃-CH₃)

(Step 8) (3′RS,4′RS)-Ethyl 11-[7-methoxymethoxy-3-(4-methoxymethoxyphenyl)-3-methylchroman-4-yl]-2-(4,4,5,5,5-pentafluoropentyl)undecanoate

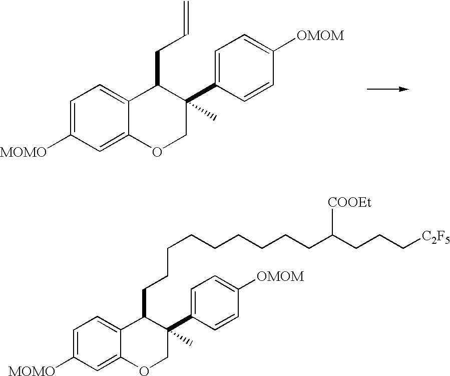

(3RS,4RS)-7-Methoxymethoxy-3-(4-methoxymethoxyphenyl)-3-methyl-4-(2-propenyl)chromane (6.9 g, 18.0 mmol) prepared in Step 7 was dissolved in dichloromethane (150 m), ethyl 2-(4,4,5,5,5-pentafluoropentyl)-9-decenoate (13.3 g, 35.2 mmol) prepared in Step 3 and benzylidene-bis(tricyclohexylphosphine)dichlororuthenium (745 mg, 0.91 mmol) were added thereto, and the mixture was refluxed overnight while stirring. The reaction mixture was concentrated under reduced pressure and purified by column chromatography (eluent: n-hexane/ethyl acetate=30/1, v/v) to give 7 g of a colorless oil. The oil thus obtained was dissolved in 250 ml of tetrahydrofuran, 10% Pd/C (1.4 g) was added thereto, and the resulting mixture was stirred overnight under hydrogen atmosphere. The reaction mixture was filtered through cellite and concentrated under reduced pressure to give 6.6 g (Yield 51%) of the pure title compound.

¹H-NMR (300 MHz, CDCl₃): δ 7.11(d, 2H, J=8.7 Hz), 7.00(d, 2H, 8.7 Hz), 6.93(m, 1H), 6.55-6.53(m, 2H), 5.15(s, 2H), 5.12(s, 2H), 4.50(d, 1H, J=10.4 Hz), 4.24(d, 11, J=10.5 Hz), 4.11(q, 2H, J=5.7 Hz), 3.47(s, 6H), 2.62(m, 1H), 2.32(m, 1H), 2.05-1.94(m, 2H), 1.68-0.95(m, 28H)

(Step 9) (3′RS,4′RS)-11-[7-Hydroxy-3-(4-hydroxyphenyl)-3-methylchroman-4-yl]-2-(4,4,5,5,5-pentafluoropentyl)undecanoic Acid

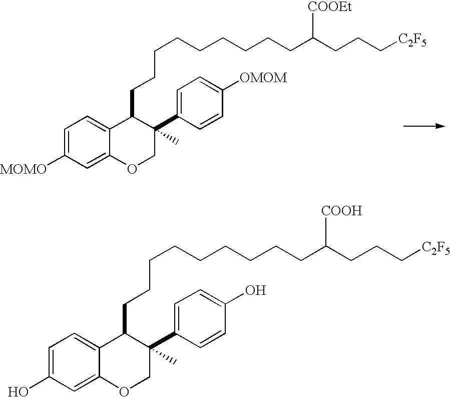

(3′RS,4′RS)-Ethyl 11-[7-methoxymethoxy-3-(4-methoxymethoxyphenyl)-3-methyl chroman-4-yl]-2-(4,4,5,5,5-pentafluoropentyl)undecanoate (6.6 g, 9.2 mmol) was dissolved in methanol (160 ml), conc. hydrochloric acid (0.5 ml) was added thereto, and the mixture was refluxed for 3 hours. The mixture was cooled to room temperature, water was added thereto, and the resulting mixture was extracted with ethyl acetate. The organic layer thus obtained was washed with water and saturated saline solution, dried over anhydrous magnesium sulfate, filtered, concentrated under reduced pressure, and purified by column chromatography (eluent: n-hexane/ethyl acetate=15/1, v/v) to give 6 g of a colorless oil. The oil thus obtained was dissolved in a solvent mixture of ethanol-water (150/50 ml), KOH (11.3 g, 0.19 mol) was added thereto, and the resulting mixture was refluxed for 3 hours. The mixture was cooled to room temperature, water was added thereto, and extracted with ethyl acetate. The organic layer thus obtained was washed with water and saturated saline solution, dried over anhydrous magnesium sulfate, filtered, concentrated under reduced pressure, and purified by MPLC (Medium Pressure Liquid Chromatography; eluent: methanol/water=4/1→5.5/1 column RP-18) to give the title compound (3.2 g, Yield 56%) as a pure oil.

¹H-NMR (300 MHz, CDCl₃): δ 7.07(d, 2H, J=8.6 Hz), 6.90(dd, 11, J1=5.7 Hz, J2=3.1 Hz), 6.83(d, 2H, J=8.6 Hz), 6.40-6.38(m, 2H), 4.51(d, 1H, J=10.4 Hz), 4.24(dd, 1H, J1=10.4 Hz, J2=1.3 Hz), 2.61(m, 1H), 2.40(m, 1H), 2.14-1.93(m, 2H), 1.76-1.48(m, 6H), 1.44-0.97(m, 19H)

(Step 10) Sodium (3′RS,4′RS)-11-[7-hydroxy-3-(4-hydroxyphenyl)-3-methylchroman-4-yl]-2-(4,4,5,5,5-pentafluoropentyl)undecanoate

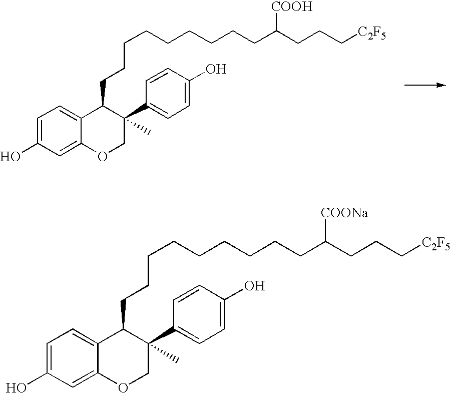

To a solution of (3′RS,4′RS)-11-[7-hydroxy-3-(4-hydroxyphenyl)-3-methylchroman-4-yl]-2-(4,4,5,5,5-pentafluoropentyl)undecanoic acid (270 mg, 0.45 mmol) in absolute methanol (2 ml) was added a solution of sodium methoxide in methanol (0.45 ml, 0.45 mmol, 1 mol/l solution, F=1.001) at room temperature, which was then stirred for 1.5 hour at the same temperature. Then, anhydrous diethylether was added to the reaction mixture and evaporated under reduced pressure to give the title compound (250 mg, Yield 89%) as a white amorphous solid.

¹H-NMR (270 MHz, CD₃OD, 3RS,4RS-compound): δ 7.02 (d, J=8.6 Hz, 2H, Ar—H), 6.79 (d, J=8.3 Hz, 1H, Ar—H), 6.73 (d, J=8.6 Hz, 2H, Ar—H), 6.26 (dd, J=8.3 Hz and 2.3 Hz, 1H, Ar—H), 6.18 (d, J=2.3 Hz, 1H, Ar—H), 4.44 (d, J=10 Hz, 1H, C2-H), 4.13 (d, J=10 Hz, 1H, C2-H), 2.55 (bs, J=not resolved, 1H, C4-H), 2.20-2.00 (m, 3H, alkyl-H and α-position of carboxylic acid), 1.70-1.00 (m, 25H, C₃-CH₃ and alkyl-H)

Experiment 1

Anti-Estrogenic Activity via Oral Administration

Oral anti-estrogenic activity in vivo of the test compound was determined according to the method described hereinafter. In this experiment, the compound of Example 1 was used as the test compound, and the known anti-estrogenic compound ICI182,780(see: U.S. Pat. No. 4,659,516) and the free acid compound prepared in Step 14 of Example 1 were used as the control compound.

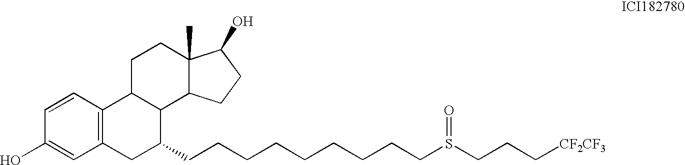

Anti-estrogenic activity was determined by subcutaneously administering 17β-estradiol-benzoate (Sigma) to mice (ICR, weight 30±2 g), which were ovariectomized 2 weeks before, in the amount of 0.1 μg/day, per mouse for 3 days and then measuring the degree that the test compound inhibits the increase in uterus weight by stimulus with estradiol. In this experiment, the test compound or the control compound was suspended in 5% arabic gum solution and orally administered for 3 days, once a day. After 24 hours from the last administration, the test animal was sacrificed and uterus was removed and weighed. The results as measured are described in the following Table 1.

TABLE 1
Anti-estrogenic activity (oral administration, 3 days)

| Test compound/dosage (p.o., 3 days) | | Inhibition (%) |
|---|---|---|
| Compound of Example 1 | 10 mg/kg | 74 |
| Free acid compound of Step 14 of Example 1 | 10 mg/kg | 79 |
| ICI182,780 | 10 mg/kg | 69 |

From the results described in the above Table 1, it could be seen that the metal salt compound according to the present invention shows an excellent inhibition activity against the increase of uterine weight by estradiol in the same manner as the free acid compound or the known ICI182,780 compound when administered per oral.

Experiment 2

Solubility Test

The degree of improvement in solubility was determined by measuring the solubility of the test compound as follows. In this experiment, the compound of Example 1 was used as the test compound, and the known anti-estrogenic compound ICI182,780 and the free acid compound prepared in Step 14 of Example 1 were used as the control compound as in Experiment 1.

To 1 mg of each compound was added 2 ml of FaSSIF (artificial intestinal juice, bile) or water, which was then shaken at 37° C. After 1, 2 and 20 hours, 200 μm of sample was taken and filtered. The concentration of the filtrate was measured by HPLC and the results are described in the following Table 2.

TABLE 2
Solubility test result

| Solubility | Hour | FaSSIF | Water |
|---|---|---|---|
| ICI182,780 | 1 | 0.365 | ND |
| | 2 | 0.474 | ND |
| | 20 | 1.699 | ND |
| Free acid compound | 1 | 10.000 | ND |
| of Step 14 of Example 1 | 2 | 17.752 | ND |
| | 20 | 86.682 | ND |
| Compound of Example 1 | 1 | 162.021 | 166.208 |
| (Na salt) | 2 | 218.555 | 187.597 |
| | 20 | 283.782 | 120.982 |

As can be seen from the results of Table 2, when the artificial intestinal juice (FaSSIF) was used as the solvent, the metal salt compound according to the present invention was observed to have a several to scores of improved solubility over the free compound or the known ICI182,780 compound. Further, when water is used as the solvent, the metal salt compound shows the same degree of excellent solubility as in the artificial intestinal juice, whereas the free compound or the known ICI182,780 compound can hardly be solved in water.

1. A compound of formula (1): 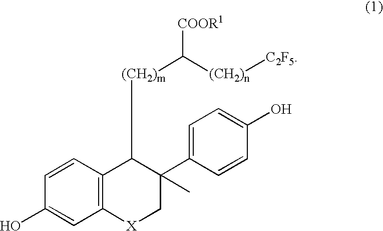
in which
X represents O or S,
R¹ represents metal,
m represents an integer of 2 to 14, and
n represents an integer of 2 to 7, stereoisomers or hydrates thereof.

2. The compound of claim 1 wherein m is an integer of 6 to 10.

3. The compound of claim 1 or 2 wherein m is an integer of 8 or 9.

4. The compound of claim 1 wherein n is an integer of 3 to 5.

5. The compound of claim 1 wherein configuration of 3- and 4-position chiral carbons in the chromane (or thiochromane) ring is (3R, 4R) or (3S, 4S) or mixtures thereof.

6. The compound of claim 5 wherein the 4-position chiral carbon of chromane (or thiochromane) ring, to which R¹OOC— group is attached, has the configuration of R or S or mixtures thereof.

7. The compound of claim 1 wherein R¹ represents alkali metal.

8. The compound of claim 7 wherein R¹ represents Na.

9. The compound of claim 8 which is selected from the group consisting of:
Sodium (3′RS,4′RS)-10-[7-hydroxy-3-(4-hydroxyphenyl)-3-methylthiochroman-4-yl]-2-(4,4,5,5,5-pentafluoropentyl)decanoate;
Sodium (3′RS,4′RS)-11-[7-hydroxy-3-(4-hydroxyphenyl)-3-methylthiochroman-4-yl]-2-(4,4,5,5,5-pentafluoropentyl)undecanoate; and
Sodium (3′RS,4′RS)-11-[7-hydroxy-3-(4-hydroxyphenyl)-3-methylchroman-4-yl]-2-(4,4,5,5,5-pentafluoropentyl)undecanoate.

10. An anti-estrogenic pharmaceutical composition which comprises effective amount of the compound of formula (1) as defined in claim 1 as an active component together with pharmaceutically acceptable carriers.

11. The anti-estrogenic pharmaceutical composition of claim 10, which is used for the treatment of breast cancer.

12. The anti-estrogenic pharmaceutical composition of claim 10 or 11, which is formulated to an oral preparation.
